(19) United States
(12) Patent Application Publication
(10) Pub. No.: US 2025/0000759 A1
(43) Pub. Date: Jan. 2, 2025

(54) EMULSION COMPOSITIONS CONTAINING HYDROPHOBICALLY-MODIFIED POLYSACCHARIDE COMPOUND AND AQUEOUS PHASE FILM FORMER

(71) Applicant: L'OREAL, Paris (FR)

(72) Inventors: Yixin YANG, Union, NJ (US); Treasure BELLAMY, Colonia, NJ (US); Brady ZARKET, Edison, NJ (US)

(21) Appl. No.: 18/345,554

(22) Filed: Jun. 30, 2023

(51) Int. Cl. A61K 8/06; A61K 8/04; A61K 8/73; A61K 8/81; A61K 8/92; A61Q 1/02

(52) U.S. Cl. CPC A61K 8/062; A61K 8/042; A61K 8/73; A61K 8/8182; A61K 8/92; A61Q 1/02; A61K 2800/42

(57) ABSTRACT

The invention relates to oil-in-water (o/w) emulsion compositions, preferably for skin, including at least one hydrophobically-modified polysaccharide compound and at least one aqueous phase film former, as well as to methods and kits including such emulsion compositions in container(s) or in application.

### FIELD OF THE INVENTION

The present invention relates to oil-in-water (o/w) emulsions (compositions) comprising at least one hydrophobically-modified polysaccharide compound and at least one aqueous phase film former, as well as to methods and kits comprising such emulsion compositions in container(s) or in application. The compositions have beneficial cosmetic properties including, for example, as primer or setter compositions for good or improved wear, water and/or sebum resistance, and/or transfer resistance properties of separately-applied compositions.

### DISCUSSION OF THE BACKGROUND

At the present time on the market for caring for and making up keratin materials, many products claim staying power throughout the day, withstanding external factors such as water, sebum, mechanical friction, etc. (waterproof mascara, food-proof lipsticks, long-lasting foundations). Long-lasting products for the lips, the eyelashes, the eyebrows or the face, which can be used at home, are mainly based on synthetic coating polymers in the presence of organic solvents. For making up the lips and compositions for making up the face, compositions comprising unmodified silicone resin as film forming agent are known, such as the compound having the INCI name: Trimethylsiloxysilicate or a compound having the INCI name: Polypropylsilsesquioxane, or alternatively a silicone acrylate copolymer such as the product having the INCI name: Acrylates/polytrimethylsiloxymethacrylate copolymer). Long-lasting makeup products for the eyelashes and/or the eyebrows (mascaras, eyeliners) use, for their part, typically use waxes or film-forming polymer particles in aqueous suspension of the latex type (i.e.: styrene/acrylate copolymers) as film formers.

Besides these long-lasting products, the current trend is towards semi-permanent makeup. Specifically, in recent years, conventional makeup products have met with competition from the market of semi-permanent makeup in professional salons. It is encountered in the sector of makeup for the eyes (semi-permanent mascara, permanent eyelash makeup, eyelash extensions, etc.), for the eyebrows (semi-pigmentation known as micro-blading), for the complexion (freckles, beauty spots or the whole face, glowing or healthy-complexion effect) or for the lips (semi-permanent tattooing). This new trend is driving consumers towards seeking increasingly long staying power for greater practicality (avoiding having to apply and remove makeup daily, healthy complexion effect immediately on waking up, etc.).

The aim of the present invention is to propose compositions which offer excellent staying power of the expected cosmetic effects, notably the color of the makeup on keratin materials (skin, lips, nails, hair, eyelashes, eyebrows) which may extend the duration of the cosmetic composition on keratin materials, as well as improve wear of the cosmetic composition on keratin materials and its resistance to external forces such as one or more of mechanical friction, water, sweat and perspiration, sebum, oil, etc.

Possibly related patent references include:
U.S. Pat. No. 11,253,462 discloses low-wax compositions containing silicone polymers among other ingredients;
US 2020/0022898 discloses film forming compositions comprising a first anionic or non-ionic non-crosslinking polyacrylate random copolymer and a second cationic non-crosslinking polyamide random copolymer;
U.S. Pat. No. 11,033,478 discloses a self-curling mascara composition containing at least about 7 percent by weight of a non-ionic water-soluble or water dispersible copolymer that includes a cyclic amide monomer, a cyclic amine monomer, and an acrylamide monomer;
US 2003/0082221 discloses compositions containing pullulan;
US 2016/0113860 discloses compositions containing silicone-modified pullulan and silicone oils which it thickens;
JP-A H08-208989 is discussed in the previous application, and characterized as disclosing silicone-modified pullulan which thickens silicone oil components, but which has an inadequate thickening effect;
CN 111801092 discloses compositions for impregnating a material, where the compositions contain PEG or polyethyer modified-silicones as well as dendrimer type silioxanes;
FR 3,104,958 discloses compositions containing polyurethane dispersions among other ingredients; and
FR 3,110,849 discloses compositions containing polyurethane and/or acrylic dispersions among other ingredients, with little or no fatty material.

There remains a need for improved compositions such as primer or setter compositions having improved properties with respect to composition ease of and/or comfort of application of the composition, and/or in particular with respect to improved properties related to improved wear of the cosmetic composition on keratin materials and its resistance to external forces such as mechanical friction, water, sweat and perspiration, sebum and/or oil.

### SUMMARY OF THE INVENTION

The present invention relates to oil-in-water (o/w) emulsions (compositions) comprising at least one hydrophobically-modified polysaccharide compound and at least one aqueous phase film former. Preferably, the composition is a composition for application to skin. Most preferably, the composition is a setter or a primer composition, and/or is in the form of a gel. Also preferably, the composition further comprises at least one gelling agent and/or at least one active agent.

The present invention also relates to methods of setting a color cosmetic composition (e.g., composition containing colorant such as a foundation) and/or-priming keratinous material for a color cosmetic composition by applying compositions of the present invention to the keratinous material (priming) and/or to a color cosmetic composition previously-applied to keratinous material (setting) in an amount sufficient to prime the keratinous material for the color cosmetic composition and/or in an amount sufficient to obtain setting of the previously-applied color cosmetic composition to keratinous material.

The present invention also relates to kits comprising, as separate compositions in one or more containers within the kits, (A) at least one composition of the present invention, in particular a setter or primer composition for cosmetics, comprising at least one hydrophobically-modified polysaccharide compound and at least one aqueous phase film former; and (B) at least one cosmetic composition comprising at least one colorant.

The present invention also relates to sets, as applied onto keratinous material, comprising (A) at least one first layer of at least one cosmetic composition comprising at least one colorant; and (B) at least one second layer of at least one composition of the present invention, in particular a setter or primer composition for cosmetic compositions comprising at least one colorant, comprising at least one hydrophobically-modified polysaccharide compound and at least one aqueous phase film former. Preferably, the at least one second layer is applied over the at least one first layer (in which case the second layer is a setter layer formed by application of a setter composition to the first layer). However, if the at least one first layer is applied over the at least one second layer, the at least one second layer is a primer layer formed by application of a primer composition over keratinous material.

The present invention also relates to methods of making oil-in-water (o/w) emulsions (compositions) of the present invention comprising combining at least one hydrophobically-modified polysaccharide compound and at least one aqueous phase film former during formation of the emulsion composition. Preferably, the composition is a composition for application to skin. Most preferably, the composition is a setter or a primer composition, and/or is in the form of a gel. Also preferably, the composition further comprises at least one gelling agent and/or at least one active agent.

It is to be understood that both the foregoing general description and the following detailed description are exemplary and explanatory only, and are not restrictive of the invention.

### DETAILED DESCRIPTION OF THE INVENTION

In the following description of the invention and the claims appended hereto, it is to be understood that the terms used have their ordinary and accustomed meanings in the art, unless otherwise specified.

“About” as used herein means within 10% of the indicated number (e.g., “about 10%” means 9%-11% and “about 2%” means 1.8%-2.2%).

“A” or “an” as used herein means “at least one.”

“At least one” means one or more and thus includes individual components as well as mixtures/combinations.

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc.

“Film former”, “film-forming polymer” or “film-forming agent” as used herein means a polymer or resin that leaves a film on the substrate to which it is applied, for example, after a solvent accompanying the film former has evaporated, absorbed into and/or dissipated on the substrate.

“Wax” as used herein is a lipophilic fatty compound that is solid at ambient temperature (25° C.) and changes from the solid to the liquid state reversibly, having a melting temperature of more than 30° C. and, for example, more than 45° C., and a hardness of more than 0.5 MPa at ambient temperature.

“Surfactant” and “emulsifier” are used interchangeably throughout this specification.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 115° C.

“Non-volatile”, as used herein, means having a flash point of greater than about 115° C.

“Polymer” as used herein means a compound which is made up of at least two monomers.

“Matte” in compositions as used herein refers to compositions having little to no light reflection. For example, matte compositions can have average gloss properties, measured at 60°, of less than or equal to 10, for example 9, preferably less than or equal to 8, 6, 5, 4 or 1, including all ranges and subranges therebetween such as 1-10, 1-5, 2-10, 3-8, less than 1, less than 3, less than 5, etc. Such measurements can be made by depositing films to be tested onto a substrate (ex. a black scrub panel P121-10N or opacity card BYK No. 2810) using a drawdown bar (for example, of 1 mil, 3 mil, or 6 mil thickness) and an Automatic Drawdown Machine. The films can then be dried at room temperature and analyzed using a gloss meter (BYK: micro-TRI-gloss) at an angle of 60°.

“Free” or “substantially free” or “devoid of” as it is used herein means that while it is preferred that no amount of the specific component be present in the composition, it is possible to have very small amounts of it in the compositions of the invention provided that these amounts do not materially affect at least one, preferably most, of the advantageous properties of the conditioning compositions of the invention. Thus, for example, “free of colorant” means that an effective amount (that is, more than trace amounts) of colorant is omitted from the composition (that is, about 0% by weight), “substantially free of colorant” means that colorant is present in amounts not greater than 0.1% by weight, and “devoid of colorant” means that colorant is present in amounts not greater than 0.25% by weight, based on the total weight of the composition. The same nomenclature applies for all other ingredients identified throughout the application and in this paragraph such as, for example, silicone oils (compositions of the invention which are “free of silicone oils,” “substantially free of silicone oils,” and “devoid of silicone oils” have meanings consistent with the discussion within this paragraph), even if not specifically discussed for each identified ingredient. Thus, for example, “free of phenylated silicone oil” means that an effective amount (that is, a matte-inhibiting effective amount) of phenylated silicone oil is omitted from the composition (that is, about 0% by weight), “substantially free of phenylated silicone oil” means that phenylated silicone oil is present in amounts not greater than 0.25% by weight, and “devoid of phenylated silicone oil” means that phenylated silicone oil is present in amounts not greater than 0.5% by weight, based on the total weight of the composition. The same nomenclature applies for all other ingredients identified throughout the application and in this paragraph such as, for example, (1) mattifying fillers (compositions of the invention which are “free of mattifying fillers,” “substantially free of mattifying fillers,” and “devoid of mattifying fillers,”) and (2) additional film formers other than hydrophobically-modified polysaccharide compound and aqueous phase film former (compositions of the invention which are “free of additional film formers,” “substantially free of additional film formers,” and “devoid of additional film formers”) have meanings consistent with the discussion within this paragraph, even if not specifically discussed for each identified ingredient. Discussed examples of the use of such language such as those in this paragraph are intended to be exemplary, not limiting.

“Makeup Result” as used herein, refers to compositions where color remains the same or substantially the same as at the time of application, as viewed by the naked eye, after an extended period of time. “Makeup Result” may be evaluated by evaluating long wear properties by any method known in the art for evaluating such properties. For example, long wear may be evaluated by a test involving the application of a composition to keratin materials such as lips and evaluating the color of the composition after an extended period of time. For example, the color of a composition may be evaluated immediately following application to keratin materials such as lips and these characteristics may then be re-evaluated and compared after a certain amount of time. Further, these characteristics may be evaluated with respect to other compositions, such as commercially available compositions.

“Keratinous material” or “keratin material” means natural nails, lips, skin such as the face, the body, the hands, and the area around the eyes, and keratin fibres such as head hair, eyelashes, eyebrows, bodily hair of a human, as well as synthetic additions such as false eyelashes, false eyebrows, false nails, etc.

“Physiologically acceptable” means compatible with keratinous material and having a pleasant color, odor and feel, and which does not cause any unacceptable discomfort (stinging or tautness) liable to discourage a consumer from using the composition. Compositions of the present invention may be in the form of a gel composition.

“Gel composition” means a composition which does not flow similar to a liquid when applied to a substrate, a composition which has a 3-dimensional network that inhibits the composition from spreading on, or dripping from, a substrate after application owing to gravity over a short period of time (e.g., less than 10 seconds). G′ (storage modulus) is higher than G″ (loss modulus) at all range of strains up to 300% strain. Preferably, G′ (storage modulus) is higher than G″ (loss modulus) at low strain, but with G′ decreasing and G″ increasing, and the gel has the crossover point at >0.1% strain, preferably >1% strain, and preferably less than 200% strain, preferably less than 150% strain.

“Gel Crossover Point” (Sol/Gel Point), means the point at which the G″ (loss modulus) intersects the G′ (storage modulus), reported in % strain. It is the point at which a composition goes from a more solid state to a more liquid state. An example of a method for determining gel crossover point is as follows: G″ (loss modulus) and G′ (storage modulus) using a Discovery HR-3 Rheometer by TA Instruments, having 40 mm parallel plate geometry on a stainless steel flat peltier plate. The test can be run @ 25° C., with test parameter of angular frequency of 1.0 rad/s and logarithmic sweep: Strain % 0.01 to 1000.0%. 5 points per decade. Results reported in % strain.

“Natural compound” refers to any compound derived directly from a natural substance such as a plant without having undergone any chemical modification.

“Compound of natural origin” refers to any compound derived from a natural compound which has undergone one or more chemical modifications, for example by organic synthesis reaction, without the properties of the natural compound having been modified.

“Synthetic compound” refers to any compound which is not a natural compound or a compound of natural origin.

“Room temperature” means 25° C.

“Atmospheric pressure” means 760 mmHg, i.e. 105 pascals.

The compositions and methods of the present invention can comprise, consist of, or consist essentially of the essential elements and limitations of the invention described herein, as well as any additional or optional ingredients, components, or limitations described herein or otherwise useful. For example, the aqueous phase solvent system can “consist essentially of” water and C2-C5 monoalcohol, and/or the film forming system of the composition can consist essentially of the hydrophobically-modified polysaccharide compound and the aqueous phase film former; similarly, the oil phase film forming system can consist essentially of the hydrophobically-modified polysaccharide compound.

For purposes of the present invention, the “basic and novel property” associated with compositions, components and methods which “consist essentially of” identified ingredients or actions is transfer resistance.

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

All U.S. patents or patent applications disclosed herein are expressly incorporated by reference in their entirety.

### Aqueous Phase Film Former

According to the present invention, compositions including one or more aqueous phase film-forming polymers (“first film-forming polymers” or “first film formers”) that is/are non-ionic water-soluble or water dispersible polymer are provided. For clarity, by “non-ionic water-soluble or water-dispersible” it is meant that the polymer is non-ionic. The polymer is also water-soluble or water-dispersible, particularly to the extent that it can be readily stabilized throughout a vehicle (e.g., water) present in the composition.

The one or more of these first film-forming polymers are part of a aqueous phase film-forming system. The aqueous phase film forming system can comprise, consist essentially of, or consist of one aqueous phase film former.

Examples of aqueous phase film forming polymer include, but are not limited to, homopolymers and copolymers containing at least one (meth)acrylic acid ((meth)acrylate) monomer and/or at least one vinylpyrrolidone monomer such as polyvinylpyrrolidone, vinyl pyrrolidone/vinyl acetate copolymers, vinyl pyrrolidone/acrylic acid copolymers, vinyl pyrrolidone/acrylate copolymers, alkyl (e.g., butyl) acrylate/hydroxyalkyl (e.g., propyl)dimethicone acrylate copolymers, styrene/acrylates/ammonium methacrylate copolymers, acrylamide/sodium acryloyldimethyltaurate copolymer, etc.; water-soluble polysaccharides such as pectin, unmodified pullulan or cellulose compounds; polyurethanes; latexes; and mixtures thereof.

According to preferred embodiments, the at least one aqueous phase film forming polymer is at least one cyclic amide-containing polymer. Preferably, the at least one cyclic amide-containing polymer also has at least one of cyclic amine and/or acrylamide functionality as well, preferably both of these, although it is possible for the at least one polymer to contain neither cyclic amine nor acrylamide functionality.

Cyclic amide and cyclic amine monomers useful in the preferred first film-forming polymers include those having one or more aromatic or aliphatic ring structures. These rings may have sizes ranging from about having sizes of, for example, 5 to 8 ring members.

In certain embodiments, monomers useful in forming these first film-forming polymers are polymerizable, ethylenically-unsaturated monomers having a cyclic amine residue or a cyclic amide residue. Accordingly, the cyclic amide monomers of these first film-forming polymers may include cyclic amide residues that are or include heterocyclic ring structures such as lactams and the like. These may include a-Lactam, β-lactam, γ-lactam. δ-lactam, and ε-lactam. In one preferred embodiment, the cyclic amide is a pyrrolidone (a γ-lactam), in particular vinylpyrrolidone

Useful cyclic amine residues may include any of various heterocyclic amines such as azoles, pyrroles, pyrrolidines, carbamates, and the like. In one preferred embodiment, the cyclic amine residue is an imidazole.

In certain embodiments, acrylamide monomers useful in these first film-forming polymers include those having-C3H5NO functional groups. Examples include (meth)acrylamides.

As noted above, at least one cyclic amide-containing polymer preferably has at least one of cyclic amine and acrylamide functionality as well, preferably both, although it is possible that the polymer contains neither functionality. For example, polymer(s) containing cyclic amide residue(s) but not cyclic amine or acrylamide functionality include but are not limited to vinylpyrrolidone homopolymers (polyvinylpyrrolidone) and vinylpyrrolidone copolymers containing at least one monomer other than vinylpyrrolidone such as, for example, substituted or unsubstituted C2 (acrylic acid) or C3 (allyl) groups, possibly in the form of esters (for example, acrylates or methacrylates) or ethers.

In certain embodiments, the at least one aqueous phase film forming polymer has a weight average molecular weight in a range from about 10,000 daltons to about 1,000,000 daltons, including all ranges and subranges therebetween such as, for example, 50,000 daltons to 500,000 daltons, 75,000 daltons to 300,000 daltons, and 100,000 daltons to 250,000 daltons.

In certain embodiments, the at least one cyclic amide-containing polymer is a copolymer of N-vinyl pyrrolidone, methacrylamide, and N-vinylimidazole.

In certain embodiments, the at least one cyclic amide-containing polymer may be a commercially available variety, such as LUVISET CLEAR AT3, commercially available from BASF of Ludwigshafen, Germany.

According to preferred embodiments, the compositions of the present invention are devoid of, free of, or substantially free of, polyurethanes.

Preferably, the at least one aqueous phase film forming polymer is present in the compositions of the present invention in an amount ranging from about 0.5% to about 25% by weight, preferably from about 0.75% to about 20% by weight, preferably from 1% to about 10%, and preferably from about 1% to about 5% by weight with respect to the weight of the composition, including all ranges and subranges therebetween such as, for example, 4% to 15%, 1% to 3%, 3% to 25%, etc. Preferably, the at least one aqueous phase film forming polymer is present in the compositions of the present invention in an amount greater than 1% by weight of the total weight of the composition.

### Hydrophobically-Modified Polysaccharide Compound

According to the present invention, compositions including one or more hydrophobically-modified polysaccharide compound(s) (“second film-forming polymers” or “second film formers”) are provided. The one or more of these second film-forming polymers are part of a composition film-forming system along with the at least one aqueous phase film former. Optionally, other film forming agents can be in the composition film-forming system in the composition, if desired. Preferably, the at least one hydrophobically-modified polysaccharide is at least one hydrophobically-modified pullulan compound.

Pullulan is a natural polysaccharide produced from starch by the fungus Aureobasidium pullulans. Pullulan is an alpha-glucan mainly constituted of maltotriose repeating units linearly joined through alpha 1,6-glycosidic linkages. Natural pullulan is readily soluble in cold or warm water and forms clear, viscous solutions in it.

Pullulan may be produced, for example, from the fermentation of partially hydrolyzed starch or by using sucrose, sugar cane, milk, potato and other sources for carbohydrate starting materials.

Suitable pullulan compounds include those described in U.S. patent application 2018/0133144, the entire contents of which is incorporated herein by reference.

Preferably, pullulan compounds of the present invention are oil-soluble. For example, preferred pullulan compounds of the present invention have been modified with a hydrophobic group such as a silicone group or a hydrocarbon group which imparts oil-solubility to the compound. Such modifications can include, for example, a silicone group such as a siloxysilyl group (-[—SiRR′—SiOR″R″′—]-), where R, R′, R″ and R′″ are alkyl groups preferably containing 1 to 8 carbon atoms, preferably 1 to 4 carbon atoms and preferably 1 to 3 carbon atoms), or the can include C8 or greater hydrocarbon groups (for example, hydrocarbon groups containing 8 to 24 carbon atoms including ranges and subranges therebetween such as 12 to 22 carbon atoms and 12 to 18 carbon atoms).

Preferably, pullulan may be modified with a silicone group which imparts oil solubility to the compound (“silicone-modified pullulan compound.”) A preferred silicone-modified pullulan compound has a structure corresponding to:
pullulan-linker-silicone group;
where the silicone group is preferably a C1-C6 alkyl-substituted siloxysilyl group such as, for example, a monoalkyl-substituted siloxysilyl group, a diakyl-substituted siloxysilyl group, or a trialkyl-substituted siloxysilyl group, where the C1-C6 alkyl group is linear, cyclic or branched, substituted or unsubstituted; and
where the linker does not adversely affect either the film forming properties or the oil-solubility properties of the compound. Preferably, the linker is alkyl (—C—), ester or ether (—O—), amide or amine (—N—) or a combination thereof such as a carbamoyl group. Preferably, the linker is a carbamoyl group.

Preferably, the pullulan compound is a trialkylsiloxysilylcarbamoyl pullulan, with a particularly preferred compound being trimethylsiloxysilylcarbamoyl pullulan.

Preferably, pullulan compound(s) in the compositions of the present invention have a molecular weight of 50,000 to 10,000,000 daltons, preferably 100,000 to 5,000,000 daltons, preferably 250,000 to 1,000,000 daltons, including all ranges and subranges therebetween including, for example, 200,000 to 7,600,000 daltons, 350,000 to 8,750,000 daltons, 225,000 to 700,000 daltons, etc.,

Preferably, the at least one hydrophobically-modified polysaccharide compound is present in the compositions of the present invention in an amount ranging from about 0.5% to about 25% by weight, preferably from about 0.75% to about 20% by weight, preferably from about 1% to about 10%, and preferably from about 1% to about 5% by weight with respect to the weight of the composition, including all ranges and subranges therebetween such as, for example, 4% to 14%, 1% to 5%, 1.5% to 8%, etc. Preferably, the at least one hydrophobically-modified polysaccharide compound is present in the compositions of the present invention in an amount greater than 1.5% by weight of the total weight of the composition.

According to preferred embodiments, the combined, total amount of aqueous phase film former and hydrophobically-modified polysaccharide compound present in the compositions of the present invention is from about 0.5% to about 25% by weight, preferably from about 0.75% to about 20% by weight, and preferably from about 1% to about 10% by weight, with all weights being based on the total weight of the composition, including all ranges and subranges therein such as, for example, 1%-10%, 2% to 8%, 2.5% to 6%; 2.75% to 8%, 1.5% to 4%, 1.25% to 5%, etc.

According to preferred embodiments, the at least one aqueous phase film former and the at least one hydrophobically-modified polysaccharide compound are present in the compositions of the present invention in a weight ratio of about 5:1 to about 1:5, preferably about 3:1 to about 1:3, and preferably about 2.5:1 to about 1:2.5, including all ranges and subranges within these ranges, and in particular ranges and subranges obtained by using the end points of the compositions in the examples below such as, for example, 3.75/1.5 (example 3-G) to 3/1.25 (example 3-1), etc.

### Aqueous Phase

According to preferred embodiments of the present invention, compositions comprising an aqueous phase solvent system comprising (i) water in an amount of at least about 10% by weight with respect to the total weight of the composition; and (ii) optionally, at least one C2-C5 monoalcohol are provided. Preferably, the composition is in the form of an emulsion with a continuous outer phase which is aqueous, preferably an oil-in-water (o/w) emulsion.

According to preferred embodiments, the compositions of the present invention comprise water. Preferably, the compositions comprise at least about 10% water by weight, preferably greater than 20% water by weight, preferably greater than about 25% water by weight, and preferably greater than about 30% water by weight, preferably in amounts from about 10% to about 80%, preferably from greater than 20% to about 75%, preferably from about 25% to about 60%, and preferably from about 30% to about 50%, by weight, based on the total weight of the composition, including all ranges and subranges therebetween such as, for example, about 30% to about 60%.

Suitable C2-C5 monoalcohols, if present, include ethanol, propanol, butanol, pentanol, isopropanol, isobutanol and isopentanol. Ethanol is particularly preferred.

Preferably, the C2-C5 monoalcohol(s), if present, is/are present in the compositions of the present invention in an amount ranging from about 1% to about 50%, preferably from about 2.5% to about 40%, preferably from about 2.5% to about 30%, and preferably from about 2.5% to about 25%, by weight, based on the total weight of the composition, including all ranges and subranges in between such as, for example, about 1% to about 25%.

Preferably, the aqueous phase solvent system of the compositions of the present invention consists essentially of, or consists of, water and, optionally, C2-C5 monoalcohols. Preferably, the aqueous phase solvent system is “free of,” “devoid of” or “substantially free of” solvents other than water and, optionally, C2-C5 monoalcohols.

### Oil Phase

According to the present invention, compositions comprising at least one oil are provided. Compositions of the present invention preferably comprise sufficient oil to form an oil-in-water emulsion, preferably from about 1% to about 80% oil, more preferably from about 2% to about 60% oil, preferably from about 5% to about 50% oil, preferably about 10% to about 45% oil, and preferably from about 20% to about 40% oil by weight with respect to the total weight of the composition, including all ranges and subranges therebetween such as, for example, 5% to 15%, 10% to 25%, etc. However, compositions “free of,” substantially free of” or “devoid of” (as defined above) oils can also be prepared.

Suitable oils include volatile and/or non-volatile oils. Such oils can be any acceptable oil including but not limited to silicone oils and/or hydrocarbon oils.

According to certain embodiments, the oil carrier comprises one or more volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |

Further, a volatile linear silicone oil may be employed in the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to other embodiments, the oil carrier comprises one or more non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, and for example, the oils sold under the trade names of Isopar or Permethyl. Preferably, the volatile non-silicone oils have a flash point of at least 40° C.

Non-limiting examples of volatile non-silicone volatile oils are given in Table 2 below.

TABLE 2

| Compound | Flash Point (° C.) |
|---|---|
| Isododecane | 43 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |
| Undecane | 62 |
| Tridecane | 94 |
| Isohexadecane | 96 |

The volatility of the solvents/oils can be determined using the evaporation speed as set forth in U.S. Pat. No. 6,338,839, the contents of which are incorporated by reference herein.

According to other embodiments of the present invention, the oil carrier comprises at least one non-volatile oil. Examples of non-volatile oils that may be used in the present invention include, but are not limited to, polar oils such as:
hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;
synthetic oils or esters of formula R5COOR6 in which R5 represents a linear or branched higher fatty acid residue containing from 1 to 40 carbon atoms, including from 7 to 19 carbon atoms, and R6 represents a branched hydrocarbon-based chain containing from 1 to 40 carbon atoms, including from 3 to 20 carbon atoms, such as, for example, Purcellin oil (cetostearyl octanoate), isononyl isononanoate, C12 to C15 alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;
synthetic ethers containing from 10 to 40 carbon atoms;
C8 to C26 fatty alcohols, for instance oleyl alcohol, cetyl alcohol, stearyl alcohol, and cetearly alcohol; and
mixtures thereof.

Further, examples of non-volatile oils that may be used in the present invention include, but are not limited to, non-polar oils such as branched and unbranched hydrocarbons and hydrocarbon waxes including polyolefins, in particular Vaseline (petrolatum), paraffin oil, squalane, squalene, hydrogenated polyisobutene, hydrogenated polydecene, polybutene, mineral oil, pentahydrosqualene, and mixtures thereof.

According to preferred embodiments, the compositions of the present invention are devoid of, free of, or substantially free of, silicone oils.

According to preferred embodiments, the compositions of the present invention are devoid of, free of, or substantially free of, non-volatile oils.

According to preferred embodiments, the compositions of the present invention are devoid of, free of, or substantially free of, waxes.

### High HLB Surfactant

According to preferred embodiments of the present invention, compositions comprising at least one high HLB surfactant are provided. “HLB” refers to the “hydrophilic-lipophilic balance” associated with emulsifiers/surfactants. In particular, “HLB” value relates to the ratio of hydrophilic groups and lipophilic groups in emulsifiers, and also relates to solubility of the emulsifiers. Lower HLB emulsifiers are more soluble in oils (lipophilic material) and are more appropriate for use in water-in-oil (W/O) emulsions. Higher HLB emulsifiers are more soluble in water (hydrophilic material) and are more appropriate for oil-in-water (O/W) emulsions. In the context of the present invention, “high HLB surfactant” means a surfactant having an HLB value of greater than 8. Also, in the context of the present invention, “high HLB value” means a surfactant having an HLB value greater than or equal to 8, preferably greater than or equal to 9, preferably greater than or equal to 10, preferably greater than or equal to 12, and preferably greater than or equal to 13.

Examples of surfactants include, but are not limited to, the following:

Polyethylene glycolated (PEG) and/or glycerolated esters or ethers, such as polyethylene glycolated and/or glycerolated branched or linear C8-C24 compounds, for example, Oleth-50, Oleth-20, Oleth-10, Ceteth-10, Steareth-20, Laureth-23, PEG-8 stearate, PEG-8 isostearate, PEG-20 stearate PEG-40 stearate, etc.;

sorbitan esters or ethers, such as oxyethylenated or nonoxyethylenated sorbitan mono- or polyalkyl esters or ethers, such as Polysorbate 21, Polysorbate 40, Polysorbate 80, Polysorbate 60, Polysorbate 61, sorbitan isostearate, glyceryl sorbitan isostearate, sorbitan sesquioleate, sorbitan laurate, sorbitan monopalmitate, sorbitan oleate, sorbitan trioleate, sorbitan monostearate and sorbitan tristearate, etc.;

Sugar mono- or polyalkyl esters or ethers, such as the mono- or polyalkyl esters or ethers of sugars as described in the patent U.S. Pat. No. 6,689,371. Mention may be made, for example, of methyl glucose isostearate, sucrose distearate, sucrose stearate, etc.;

Alkoxylated alkenyl succinates; and

Silicone derivatives, such as dimethicone copolyols, for example the mixture of cyclomethicone and of dimethicone copolyol sold under the name “DC 5225 C” by Dow Corning, and alkyl dimethicone copolyols, such as lauryl methicone copolyol, sold under the name “Dow Corning 5200 Formulation Aid” by Dow Corning, and cetyl dimethicone copolyol, sold under the name “Abil EM 90” by Goldschmidt, or the polyglyceryl-4 isostearate/cetyl dimethicone copolyol/hexyl laurate mixture sold under the name “Abil WE 90” by Goldschmidt.

The at least one high HLB surfactant is preferably present in the compositions of the present invention in an amount of about 0.1% to about 5%, preferably from about 0.1% to about 4%, and preferably from about 0.1% to about 3% by weight with respect to the total weight of the composition, including all ranges and subranges therebetween.

Gelling Agent/Colorant/Active Agent Gelling Agent

According to preferred embodiments of the present invention, compositions further comprising at least one gelling agent are provided. Suitable gelling agents include any gelling agent, such as for example, gums, clays and acrylic acid (co) polymers, such as high molecular weight homo- or co-polymers comprising acrylic acid, optionally crosslinked with a polyalkenyl polyether, including some polymers identified as “carbomer” as well as amphiphilic polymers. Preferably, the gelling agent is a gum, an amphiphilic polymer or a clay, or a combination of any of these.

According to preferred embodiments, the at least one gelling agent is preferably at least one gum. Suitable examples of thickening gums include, but are not limited to, Suitable gums include xanthan, sclerotium, pectin, karaya, arabic, gelatin, agar, guar, carrageenan, alginate and combinations thereof.

According to preferred embodiments, the at least one gelling agent is preferably at least one amphiphilic polymer, comprising at least one ethylenically unsaturated monomer, preferably containing a sulphonic group, in freeform or partially or totally neutralized form.

The amphiphilic polymers may comprise at least one hydrophobic portion. The hydrophobic portion present in these polymers preferably contains from 6 to 50 carbon atoms, preferably from 6 to 22 carbon atoms, preferably from 6 to 18 carbon atoms and preferably from 12 to 18 carbon atoms, including all ranges and subranges therebetween.

The amphiphilic polymers may have a molar mass ranging from 50,000 g/mole to 10,000,000 g/mole, preferably from 80,000 g/mole to 8,000,000 g/mole, and preferably from 100,000 g/mole to 7,000,000 g/mole.

The amphiphilic polymers may be based on at least one ethylenically unsaturated hydrophilic monomer A and on at least one hydrophobic monomer B. Preferably, the monomer A comprises a strong acid function, in particular a sulphonic acid or phosphonic acid function. The hydrophobic monomer B comprises at least one hydrophobic radical, chosen from: saturated or unsaturated C6-C18 linear alkyl radicals (for example, n-hexyl, n-octyl, n-decyl, n-hexadecyl, n-dodecyl or oleyl);

branched alkyl radicals (for example, isostearic) or cyclic alkyl radicals (for example, cyclododecane or adamantane); C6-C18 fluoro or alkylfluoro radicals (for example, the group of formula-(CH2) 2-(CF2) 9—CF3); a cholesteryl radical or radicals derived from cholesterol (for example, cholesteryl hexanoate); aromatic polycyclic groups, for instance naphthalene or pyrene; and silicone or alkylsilicone or alkylfluorosilicone radicals. Among these radicals, linear and branched alkyl radicals are preferred.

The amphiphilic polymers may be water-soluble or water-dispersible in neutralized form.

The amphiphilic polymers may be crosslinked. The crosslinking agents may be chosen from, for example, the polyolefinically unsaturated compounds commonly used for crosslinking polymers obtained by free-radical polymerization. According to one preferred embodiment of the invention, the crosslinking agent is chosen from methylenebisacrylamide, allyl methacrylate or trimethylolpropane triacrylate (TMPTA). The degree of crosslinking preferably ranges from 0.01 mol % to 10 mol %, and preferably from 0.2 mol % to 2 mol %, relative to the polymer, including all ranges and subranges therebetween.

The amphiphilic polymers may be homopolymers or copolymers.

The amphiphilic polymers can be partially or totally neutralized with a mineral base (for example, sodium hydroxide, potassium hydroxide or aqueous ammonia) or an organic base such as monoethanolamine, diethanolamine, triethanolamine, aminomethylpropanediol, N-methylglucamine, or basic amino acids, for instance arginine and lysine, and mixtures thereof.

The amphiphilic polymers may be water-soluble or water-dispersible homopolymers such as, for example, optionally cross-linked polymers of sodium 2-acrylamido-2-methylpropane sulfonate acid such as that used in the commercial product SIMULGEL 800 (CTFA name: Sodium Polyacryloyldimethyl Taurate), cross-linked polymers of ammonium 2-acrylamido-2-methyl propane sulfonate acid (INCI name: AMMONIUM POLYACRYLDIMEHYLTAURAMIDE) such as the product sold under the tradename HOSTACERIN AMPS@ by Clariant.

The amphiphilic polymers may be chosen from crosslinked or non-crosslinked amphiphilic polymers of 2-acrylamido-2-methylpropanesulphonic (AMPS) acid and of at least one ethylenically unsaturated monomer comprising at least one hydrophobic portion containing from 6 to 30 carbon atoms, preferably from 6 to 22 carbon atoms, preferably from 6 to 18 carbon atoms and preferably from 12 to 18 carbon atoms, including all ranges and subranges therebetween.

Suitable examples of amphiphilic polymers include, but are not limited to, hydrophobically-modified sulfonic acid copolymers such as Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant) (crosslinked ethoxylated AMPS/behenyl methacrylate), Ammonium Acryloyldimethyltaurate/Steareth-25 Methacrylate Crosspolymer (Aristoflex HMS) (ethoxylated copolymer of AMPS/stearyl methacrylate crosslinked with trimethylol triacrylate), Aristoflex SNC (crosslinked ethoxylated AMPS/C16-C18), Aristoflex LNC (noncrosslinked AMPS/C12-C14), acrylamide/sodium acryloyldimethyl taurate copolymer/isohexadecane/polysorbate 80) (Simulgel 600) and mixtures thereof.

According to preferred embodiments, the gelling agent(s) may be at least one clay. Among the clays, mention may be made of clays of the smectite family, such as laponite, of the kaolinite family, such as kaolinite, dickite, nacrite, optionally modified clays of the halloysite, dombassite, antigorite, berthierine, pyrophyllite, montmorillonite, beidellite, vermiculite, talc, stevensite, hectorite, saponite, chlorite, sepiolite and illite family.

Clays are products that are already well known per se, which are described, for example, in the publication “Mineralogie des argiles” [“Clay Mineralogy”], S. Caillère, S. Hénin, M. Rautureau, 2nd Edition 1982, Masson, the teaching of which is included herein by way of reference.

Natural clay is a sedimentary rock composed to a large extent of specific minerals, silicates generally of aluminium. Kaolin is thus a natural clay.

The clays may also be synthetic. Thus, Sumecton mentioned below is a synthetic saponite.

Preferably, in the context of the present invention, use is made of clays that are cosmetically compatible and acceptable with keratin materials. Clays that may especially be mentioned include kaolinite, montmorillonites, saponites, laponites, hectorites, and illites. Mixtures of clays and natural clays may also be used.

Natural clays that may be mentioned include green clays, in particular rich in illite; clays rich in montmorillonite, known as fuller's earth, or such as bentonite or else white clays rich in kaolinite. As bentonites, mention may in particular be made of those sold under the names Bentone 38 VCG®, Bentone Gel CAO VR, Bentone 27 VR and Bentone Gel MIO V® by the company Elementis.

By way of clay rich in montmorillonite, mention may be made of the aluminium silicate hydrate sold under the name Gel White H® by the company Rockwood.

By way of saponite, which belongs to the montmorillonite family, mention may be made of synthetic saponite, in particular the product sold by the company Kunimine under the name Sumecton®.

By way of tack, mention may be made of those sold under the names Rose Talc® and Talc SG-2000@ sold by the company Nippon Talc, Luzenac Pharma MR sold by the company Luzenac, J-68BC® from US Corporation and Micro ACE-P-3@ sold by the company Nippon Talc.

Preferably, if present, the gelling agent(s) is/are present in the compositions of the present invention in amounts ranging from about 0.1 to about 30% by weight, preferably from 0.5 to 25% by weight, preferably from 1 to 20% and preferably from 2.5 to 15% by weight, all weights based on the weight of the composition as a whole, including all ranges and subranges therebetween such as, for example, 0.1 to 1.5%, 2 to 20%, 10 to 20%, etc.

### Colorant (Coloring Agent)

According to embodiments of the present invention, compositions optionally further comprising at least one coloring agent are provided. According to preferred embodiments, the at least one coloring agent, if present, is at least one surface-treated pigment. “Surface-treated pigment” means pigments that have totally or partially undergone a surface treatment of chemical, electronic, electrochemical, mechanochemical or mechanical nature, with a surface treatment agent. Preferably, the pigments are selected from inorganic pigments or inorganic/organic mixed pigments.

Surface treatment agents may be selected from the group consisting of alkyl silanes, organotitanates, halogenated phosphonates, and halogenated organosilanes. According to preferred embodiments, pigments have been surface treated with a surface treatment agent selected from the group consisting of alkoxylated alkyl silanes such as, for example, ethoxylated and/or propoxylated C2-C8 alkyl silanes, and salts thereof, organotitanates such as, for example, titanium salts of fatty acids such as, for example, C2-C8 alkylated titanium salts of C9-C24 fatty acids such as stearic acid, isostearic acid, oleic acid, cetearic acid, cetyl acid, etc., halogenated organophosphonates such as, for example, perfluoroalkyl phosphonates, and salts thereof, and halogenated organosilanes such as, for example, perfluoro C2-C8 alkyl silanes (optionally ethoxylated and/or propoxylated), and salts thereof. Specific examples of suitable surface treatment agents include (1)triethoxy caprylylsilane, (2) perfluorooctyltriethoxysilane, (3) sodium perfluorohexylethylphosphonate and (4) isopropyl titanium triisosterate.

Preferred surface treatment agents are selected from the group consisting of alkyl silanes and halogenated organosilanes. According to preferred embodiments, pigments have been surface treated with a surface treatment agent selected from the group consisting of alkoxylated alkyl silanes such as, for example, ethoxylated and/or propoxylated C2-C8 alkyl silanes, and salts thereof, and halogenated organosilanes such as, for example, perfluoro C2-C8 alkyl silanes (optionally ethoxylated and/or propoxylated), and salts thereof. Specific examples of suitable surface treatment agents include (1) triethoxy caprylylsilane, and (2) perfluorooctyltriethoxysilane.

The surface-treated pigments of the present invention can be prepared according to surface treatment techniques well known to a person of ordinary skill in the art or can be found commercially.

For example, the surface treatment agent with which the pigments are treated can be deposited on the pigments by solvent evaporation, chemical reaction between the molecules of the surface treatment agent or creation of a covalent bond between the surface treatment agent and the pigments. The surface treatment can thus be carried out, for example, by chemical reaction of a surface treatment agent with the surface of the pigments and creation of a covalent bond between the surface treatment agent and the pigments. An exemplary method is described, for example, in U.S. Pat. No. 4,578,266, the entire contents of which is hereby incorporated by reference.

The at least one surface-treated pigment, if present, preferably is present in the compositions of the present invention in an active solid content amount ranging from about 1% to about 30%, preferably from about 3% to about 25%, and preferably from about 5% to about 20%, by weight with respect to the total weight of the composition, including all ranges and subranges there between.

According to preferred embodiments of the present invention, compositions further comprising at least one unsurfaced-treated (not surface-treated) coloring agent are provided. Such coloring agents, if present, may be in addition to, or instead of, the surface-treated pigment discussed above.

According to this embodiment the coloring agent is preferably chosen from pigments which are not surface-treated, dyes, such as liposoluble dyes, nacreous pigments, and pearling agents.

Representative liposoluble dyes which may be used according to the present invention include Sudan Red, DC Red 17, DC Green 6,-carotene, soybean oil, Sudan Brown, DC Yellow 11, DC Violet 2, DC Orange 5, annatto, and quinoline yellow. The liposoluble dyes, when present, generally have a concentration ranging up to 40% by weight of the total weight of the composition, such as from 0.0001% to 30%, including all ranges and subranges therebetween.

The nacreous pigments which may be used according to the present invention may be chosen from colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, be present in the composition in a concentration ranging up to 50% by weight of the total weight of the composition, such as from 0.0001% to 40%, preferably from 0.001% to 30%, including all ranges and subranges therebetween.

The non-surface treated pigments, which may be used according to the present invention, may be chosen from white, colored, inorganic, organic, polymeric, and nonpolymeric pigments. Representative examples of mineral pigments include titanium dioxide, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the coloring agents may be present in the composition in a concentration ranging up to 50% by weight of the total weight of the composition, such as from 0.0001% to 40%, and further such as from 0.001% to 30%, including all ranges and subranges therebetween.

According to preferred embodiments, the compositions of the present invention are devoid of, free of, or substantially free of, coloring agents.

### Active Agent

According to preferred embodiments of the present invention, compositions further comprising at least one active agent are provided. Preferably, the active agent is in the aqueous phase (hydrophilic active agent), although active agents may be present in the oil phase (hydrophobic active agent).

In some embodiments, the active agent may be:
a moisturizing agent, such as a polyol such as, for example, glycerin and sugars, urea and its derivatives, such as in particular hydroxyalkyl urea, in particular hydroxyalkylurea, and mixtures thereof;
a dequamating agent, which may be a compound capable of acting either directly on desquamation by promoting exfoliation, such as B-hydroxy acids, in particular salicylic acid and its derivatives (including 5-n-octanoylsalicylic acid); a-hydroxy acids, such as glycolic acid, citric acid, lactic acid, tartaric acid, malic acid or mandelic acid; urea; gentisic acid; oligofucoses; cinnamic acid; extract of Saphora japonica; resveratrol and certain jasmonic acid derivatives; or acting on the enzymes involved in the desquamation or degradation of corneodesmosomes, glycosidases, stratum corneum chymotryptic enzyme (SCCE), or even other proteases (trypsin, chymotrypsin-like). Mention may be made of agents for chelating mineral salts: EDTA; N-acyl-N,N′N′-ethylenediaminetriacetic acid; aminosulfonic compounds and in particular (N-2-hydroxyethylpiperazine-N-2-ethane) sulfonic acid (HEPES); 2-oxothiazolidine-4-carboxylic acid (procysteine) derivatives, a-amino acid derivatives of the type such as glycine (as described in EP-0 852 949 and sodium methylglycinediacetate sold by BASF under the trade name Trilon M); honey; sugar derivatives such as O-octanoyl-6-D-maltose and N-acetylglucosamine;
a humectant;
an anti-aging agent, which may include, for example, one or more of C-beta-D-xylopyranoside-2-hydroxypropane (Pro-Xylane), retinol, peptides, caffeine, and other components that provide improvement to skin texture, any other suitable soluble/dispersible targeted active ingredient, and combinations thereof.

a mattifying agent, which may include, but is not limited to, mattifying fillers such as, for example, talc, silica, silicone elastomers, and polyamides, and waxes such as, for example, beeswax and copernicia cerifera (carnauba) wax.

an antimicrobial agent, non-limiting examples of which include 2,4,4′-trichloro-2′-hydroxydiphenyl ether (or triclosan), 3,4,4′-trichlorobanilide, phenoxyethanol, phenoxypropanol, phenoxyisopropanol, hexamidine isethionate, metronidazole and its salts, micronazole and its salts, itraconazole, terconazole, econazole, ketoconazole, saperconazole, fluconazole, clotrimazole, butoconazole, oxiconazole, sullaconazole, sulconazole, terbinafine, ciclopirox, ciclopiroxolamine, undecylenic acid and its salts, benzoyl peroxide, 3-hydroxybenzoic acid, 4-hydroxybenzoic acid, phytic acid, N-acetyl-L-cysteine acid, lipoic acid, azelaic acid and its salts, arachidonic acid, resorcinol, 2,4,4′-trichloro-2′-hydroxydiphenyl ether, 3,4,4′-trichlorocarbanalide, octopirox, octoxyglycerine, octanoylglycine, caprylyl glycol, 10-hydroxy-2-decanoic acid, dichlorophenylimidazole dioxolane and its derivatives described in patent WO 93/18743, tarnesol and phytosphingosines, and mixtures thereof;

a pigment modifying agent and/or skin lightening agents, such as double-stranded RNA oligonucleotides are useful for decreasing tyrosinase expression. Mention may also be made of ceramides, vitamin C and derivatives thereof, in particular vitamin CG, CP and 3-O ethyl vitamin C, alpha- and beta-arbutin, ferulic acid, kojic acid, resorcinol and derivatives thereof, calcium D-pantetheine sulphonate, lipoic acid, ellagic acid, vitamin

B3, phenylethyl resorcinol, for instance Symwhite 377® from the company Symrise, a kiwi fruit (Actinidia chinensis) juice sold by Gattefosse, an extract of Paconia suffructicosa root, such as the product sold by the company Ichimaru Pharcos under the name Botanpi Liquid B®, an extract of brown sugar (Saccharum officinarum), such as the extract of molasses sold by the company Taiyo Kagaku under the name Molasses Liquid, a mixture of undecylenic acid and undecylenoyl phenyl alanine, such as Sepiwhite MSHO from Seppic;

a vitamin such as a vitamin A compound such as retinol or retinoic acid, vitamin B compound such as vitamin B1, B3 or B6, a vitamin

C compound such as an ascrobyl phosphate salt (e.g., magnesium or sodium, and/or derivatives thereof;

an ultraviolet (UV) filter such as, e.g., an aminobenzoic acid derivative, a dibenzoylmethane derivative, a salicylic acid derivative, a cinnamic derivative, β,β diphenylacrylate derivative, a benzophenone derivative, benzylidene camphor derivative, and mixtures thereof. Mention may be made especially of ethylhexyl methoxycinnamate sold under the tradename UVINUL MC 800 by the company BASF, of ethylhexyl salicylate sold under the tradename NEO HELIOPAN OSO by the company SYMRISE and of octocrylene sold under the tradename NEO HELIOPAN 3030 by the company SYMRISE; and/or a combination thereof.

### Additional Additives

The composition of the invention can also comprise any additive usually used in the field under consideration. For example, additional film forming agents (in addition to hydrophobically-modified polysaccharide compound and aqueous phase film former), waxes, dispersants such as poly(l2-hydroxystearic acid), preserving agents, fragrances, fillers, antioxidants, neutralizing agents, silicone elastomers, and mixtures thereof can be added. A non-exhaustive listing of such ingredients can be found in U.S. patent application publication no. 2004/0170586, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9th ed. 2002). However, it is to be understood that preferred embodiments of the present invention include compositions which are “free,” substantially free” or “devoid” of the ingredients discussed in this paragraph such as additional film forming agents and waxes.

According to preferred embodiments, the compositions of the present invention contain a total solids content (excluding film formers) of 10% by weight or less, preferably 5% by weight or less, or preferably 3% by weight or less based on the weight of the composition. Preferably, compositions of the present invention are devoid of, free of, or substantially free of, solids (excluding film formers).

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

Needless to say, the composition of the invention should be cosmetically or dermatologically acceptable, i.e., it should contain a non-toxic physiologically acceptable medium and should be able to be applied to keratinous material of human beings.

According to preferred embodiments of the present invention, methods of caring for, and/or making up, keratinous material by applying compositions of the present invention to the keratinous material in an amount sufficient to care for, and/or to make up, the keratinous material are provided. Similarly, preferred embodiments of the present invention include methods of priming keratinous material for a cosmetic composition and/or of setting a cosmetic composition which has been previously applied to keratinous material by applying compositions of the present invention over keratinous material prior to application of a cosmetic composition (priming) and/or by applying compositions of the present invention over a cosmetic composition previously-applied to keratinous material (setting). Preferably, the cosmetic composition used in conjunction with compositions of the present invention is a color cosmetic composition.

Preferably, “making up” the keratin material includes applying at least one coloring agent to the keratin material (in either the composition itself or in a color coat composition applied either over or under the composition as described above) in an amount sufficient to provide color to the keratin material.

According to particularly preferred embodiments of the present invention, methods of making up keratinous material such as skin comprising (1) applying a color cosmetic composition such as a foundation to the keratinous material (e.g., skin), (2) allowing the color cosmetic composition to dry on the keratinous material (e.g., skin), preferably allowing it to dry for about 1 minute to about 15 minutes, preferably for about 5 minutes to 10 minutes, and (3) applying a composition of the present invention over the dried color cosmetic composition which has been applied to keratinous material (e.g., skin) are provided. Preferably, compositions of the present invention are applied over the dried color cosmetic composition in an amount sufficient to improve transfer resistance of the color cosmetic composition as compared to the transfer resistance provided by the color cosmetic composition when applied to keratinous material by itself.

According to preferred embodiments of the present invention, kits comprising, as separate compositions in one or more containers within the kits, (A) at least one composition of the present invention, in particular a setter or primer composition for cosmetics, comprising at least one hydrophobically-modified polysaccharide compound and at least one aqueous phase film former; and (B) at least one other composition such as a cosmetic composition comprising at least one colorant are provided.

Compositions (A) and (B) may be contained in different portions or sections of the same container within the kit. However, compositions (A) and (B) may also be in different containers with the kit.

According to preferred embodiments of the present invention, sets, as applied onto keratinous material, comprising (A) at least one first layer of at least one composition comprising at least one colorant; and (B) at least one second layer of at least one composition of the present invention, in particular a setter or primer composition for cosmetics, comprising at least one hydrophobically-modified polysaccharide compound and at least one aqueous phase film former are provided. Preferably, the at least one second layer is applied over the at least one first layer (in which case the second layer is a setter layer formed by application of a setter composition to the first layer). However, if the at least one first layer is applied over the at least one second layer, the at least one second layer is a primer layer formed by application of a primer composition over keratinous material.

According to a preferred embodiment of the present invention, compositions having improved cosmetic properties such as, for example, increased water-resistance, sebum-resistance and/or transfer-resistance are provided. Preferably, the composition is a composition for skin. Most preferably, the composition is a setter or primer composition. Also preferably, the composition further comprises at least one gelling agent and/or at least one active agent.

According to a preferred embodiment of the present invention, methods of making oil-in-water (o/w) emulsions (compositions) of the present invention comprising combining at least one hydrophobically-modified polysaccharide compound and at least one aqueous phase film former in the amounts and/or ratios discussed above during formation of the emulsion composition are provided. Preferably, the composition is a composition for application to skin. Most preferably, the composition is a setter or primer composition, and/or is in the form of a gel. Also preferably, the composition further comprises at least one gelling agent and/or at least one active agent.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example 1—Sample Inventive Compositions

| | Total %100.00-General | Total 100%-Preferred |
|---|---|---|
| Chemical Name/Function | % wt/wt | % wt/wt |
| Surfactants | 0.1-10% | 0.1-3% |
| Aqueous Phase Film | 0.5-20% | 1-10% |
| Former | | |
| Modified Pullulan | 0.5-20% | 1-10% |
| Compound | | |
| Oils | 1-30% | 2-20% |
| Colorant/fragrance/active | Optional | Optional |
| Water | QS | QS |
| Monoalcohol | 0-30% | 2.5-25% |
| Gelling Agent | 0.1-10% | 0.1-3% |

### Example 2—Testing Protocols

Transfer resistance can be determined as follows.

A color cosmetic composition (e.g., foundation such as

L'Oreal Paris Makeup True Match Liquid Foundation) is applied on keratinous material (e.g., skin) in a controlled amount (e.g., 0.020 gram) in a controlled area (e.g., 3 cm×3 cm square). Allow 5-15 minutes until the composition has dried.

Apply a test composition (setter) in a controlled amount (e.g., the same amount as was applied of the color cosmetic composition, such as 0.020 gram) over the applied color cosmetic composition, and allow it to dry. If a test composition (setter) is not applied over the color cosmetic, the transfer-resistance of the color cosmetic by itself (without a setter composition) can be compared to the transfer-resistance when a setter composition is used in conjunction with the color cosmetic composition.

A white cotton ball/pad swatch is then used to rub across the area an identified number of times (for example, 5 times or more) to assess transfer resistance of color cosmetic composition to the ball/pad swatch after such rubbing. Transfer resistance can be evaluated by determining the difference in the amount of color cosmetic composition transferred to the cotton ball/pad swatch, with or without the applied setting composition. Transfer resistance is evaluated on a 1 to 5 scale, with 1=minimal improvement in transfer resistance of color cosmetic composition to the cotton ball/pad swatch; 3=medium improvement in transfer resistance of color cosmetic composition to the cotton ball/pad swatch; and 5=large improvement in transfer resistance of color cosmetic composition to the ball/pad swatch, with little or no transfer of color cosmetic composition to the cotton ball/pad swatch occurring.

### Example 3—Transfer Resistance Testing

The following compositions A-J were prepared and tested as setter compositions for L′Oreal Paris Makeup True Match Liquid Foundation per the protocol in Example 2.

Examples 3A-3D were comparative examples, containing little or no hydrophobically-modified pullulan compound and/or little aqueous film former.

Examples 3E-3J were representative of the invention compositions containing the indicated amounts and/or ratios of hydrophobically-modified pullulan compound and aqueous film former.

Transfer resistance of the commercial foundation+prepared setter compositions was determined per the protocol in example 2:

| | Example | Example | Example | Example |
|---|---|---|---|---|
| | 3-A | 3-B | 3-C | 3-D |
| Trimethylsiloxysilylcarbamoyl | 0.15 | 0.30 | 0.60 | 0.00 |
| Pullulan | | | | |
| Isododecane | 4.85 | 4.70 | 4.40 | 5.00 |
| Isohexadecane | 0.32 | 0.32 | 0.32 | 0.32 |
| Sorbitan Oleate | 0.04 | 0.04 | 0.04 | 0.04 |
| Polysrobate 80 | 0.10 | 0.10 | 0.10 | 0.10 |
| Sodium acrylate/sodium | 0.60 | 0.60 | 0.60 | 0.60 |
| acryloyldimethyl taurate | | | | |
| copolymer | | | | |
| Vinyl | 0.10 | 0.20 | 0.40 | 2.50 |
| pyrrolidone/Methacrylamide/ | | | | |
| Vinyl Imidazole Copolymer | | | | |
| Ethanol | 0-25 | 0-25 | 0-25 | 0-25 |
| water | QS | QS | QS | QS |
| Oil phase film former | 0.15 | 0.30 | 0.60 | 0.00 |
| Water phase film former | 0.10 | 0.20 | 0.40 | 2.50 |
| Total film former | 0.25 | 0.50 | 1.00 | 2.50 |
| Transfer test - Transfer | 1 | 1 | 2 | 2 |
| resistance | | | | |

| | Example | Example | Example | Example | Example | Example |
|---|---|---|---|---|---|---|
| | 3-E | 3-F | 3-G | 3-H | 3-I | 3-J |
| Trimethylsiloxysilylcarbamoyl | 1.50 | 1.50 | 1.50 | 3.00 | 3.00 | 3.00 |
| Pullulan | | | | | | |
| Isododecane | 3.50 | 3.50 | 3.50 | 7.00 | 7.00 | 7.00 |
| Isohexadecane | 0.32 | 0.32 | 0.32 | 0.32 | 0.32 | 0.32 |
| Sorbitan Oleate | 0.04 | 0.04 | 0.04 | 0.04 | 0.04 | 0.04 |
| Polysrobate 80 | 0.10 | 0.10 | 0.10 | 0.10 | 0.10 | 0.10 |
| Sodium acrylate/sodium | 0.60 | 0.60 | 0.60 | 0.60 | 0.60 | 0.60 |
| acryloyldimethyl taurate | | | | | | |
| copolymer | | | | | | |
| Vinyl | 1.25 | 2.50 | 3.75 | 0.00 | 1.25 | 2.50 |
| pyrrolidone/Methacrylamide/ | | | | | | |
| Vinyl Imidazole Copolymer | | | | | | |
| Ethanol | 0-25 | 0-25 | 0-25 | 0-25 | 0-25 | 0-25 |
| water | QS | QS | QS | QS | QS | QS |
| Oil phase film former | 1.50 | 1.50 | 1.50 | 3.00 | 3.00 | 3.00 |
| Water phase film former | 1.25 | 2.50 | 3.75 | 0.00 | 1.25 | 2.50 |
| Total film former | 2.75 | 4.00 | 5.25 | 3.00 | 4.25 | 5.50 |
| Transfer test - Transfer | 3 | 5 | 5 | 5 | 5 | 5 |
| resistance | | | | | | |

As can be seen, setter compositions corresponding to the present invention resulted in superior transfer resistance when used in conjunction with the commercial foundation product as compared to the comparative setter compositions containing no or too little of the hydrophobically-modified pullulan compound and/or aqueous film former.

### Example 4

Similar to example 3, the following invention setter compositions A-I were prepared and used as setter compositions for L′Oreal Paris Makeup True Match Liquid Foundation per the protocol in Example 2 to determine transfer resistance:

| | Example | Example | Example | Example | Example |
|---|---|---|---|---|---|
| | 4-A | 4-B | 4-C | 4-D | 4-E |
| Trimethylsiloxysilylcarbamoyl Pullulan | 1.50 | 1.50 | 1.50 | 1.50 | 1.50 |
| Isododecane | 3.50 | 3.50 | 3.50 | 3.50 | 3.50 |
| Isohexadecane | 0.32 | 0.32 | 0.32 | 0.32 | 0.32 |
| Sorbitan Oleate | 0.04 | 0.04 | 0.04 | 0.04 | 0.04 |
| Polysrobate 80 | 0.10 | 0.10 | 0.10 | 0.10 | 0.10 |
| Sodium acrylate/sodium acryloyldimethyl | 0.60 | 0.60 | 0.60 | 0.60 | 0.60 |
| taurate copolymer | | | | | |
| Polyvinyl pyrrolidone | 1.50 | 0 | 0 | 0 | 0 |
| Polyvinyl alcohol | 0 | 1.50 | 0 | 0 | 0 |
| Vinyl pyrrolidone/vinyl acetate copolymer | 0 | 0 | 1.50 | 0 | 0 |
| Vinyl | 0 | 0 | 0 | 1.50 | 0 |
| pyrrolidone/Dimethylaminoethylmethacrylate | | | | | |
| Copolymer | | | | | |
| Vinyl pyrrolidone/Methacrylamide/Vinyl | 0 | 0 | 0 | 0 | 1.50 |
| Imidazole Copolymer | | | | | |
| Ethanol | 0-25 | 0-25 | 0-25 | 0-25 | 0-25 |
| water | QS | QS | QS to | QS | QS |

| | Example | Example | Example | Example |
|---|---|---|---|---|
| | 4-F | 4-G | 4-H | 4-I |
| Trimethylsiloxysilylcarbamoyl | 1.50 | 1.50 | 1.50 | 1.50 |
| Pullulan | | | | |
| Isododecane | 3.50 | 3.50 | 3.50 | 3.50 |
| Isohexadecane | 0.32 | 0.32 | 0.32 | 0.32 |
| Sorbitan Oleate | 0.04 | 0.04 | 0.04 | 0.04 |
| Polysrobate 80 | 0.10 | 0.10 | 0.10 | 0.10 |
| Sodium acrylate/sodium | 0.60 | 0.60 | 0.60 | 0.60 |
| acryloyldimethyl taurate | | | | |
| copolymer | | | | |
| Butyl Acrylate/Hydroxypropyl | 1.50 | 0 | 0 | 0 |
| Dimethicone Acrylate Copolymer | | | | |
| Styrene/Acrylates/Ammonium | 0 | 1.50 | 0 | 0 |
| Methacrylate Copolymer | | | | |
| Pectin | 0 | 0 | 1.50 | 0 |
| Pullulan | 0 | 0 | 0 | 1.05 |
| Ethanol | 0-25 | 0-25 | 0-25 | 0-25 |
| water | QS | QS | QS | QS |
| Transfer test-Transfer resistance | 3 | 3 | 3 | 3 |

Similar to example 3, the invention setter compositions A-I demonstrated superior transfer resistance when used in conjunction with the commercial foundation product as compared to the comparative setter compositions containing no or too little of the hydrophobically-modified pullulan compound and/or aqueous film former.

What is claimed is:

1. An oil-in water emulsion composition comprising (a) water, (b) at least one oil, (c) at least one hydrophobically-modified polysaccharide compound; and (d) at least one aqueous phase film former.

2. The composition of claim 1, in the form of a gel.

3. The composition of claim 1, wherein the composition further comprises (e) at least one compound selected from the group consisting of at least one gelling agent, at least one colorant, at least one active agent, and mixtures thereof.

4. The composition of claim 3, wherein at least two of gelling agent, colorant and active agent are present in the composition.

5. The composition of claim 1, wherein the at least one hydrophobically-modified polysaccharide compound is a silicone-modified pullulan compound, preferably trimethylsiloxysilylcarbamoyl pullulan.

6. The composition of claim 1, wherein the at least one aqueous phase film former is selected from the group consisting of polyvinylpyrrolidone, vinylpyrrolidone/vinyl acetate copolymer, vinylpyrrolidone/acrylic acid copolymer, polyvinyl alcohol, copolymer of N-vinyl pyrrolidone, methacrylamide, and N-vinylimidazole, polyurethanes, latexes, pectin, water-soluble cellulose compounds, and mixtures thereof.

7. The composition of claim 1, wherein the at least one aqueous phase film former is a copolymer of N-vinyl pyrrolidone, methacrylamide, and N-vinylimidazole.

8. The composition of claim 5, wherein the at least one aqueous phase film former is a copolymer of N-vinyl pyrrolidone, methacrylamide, and N-vinylimidazole.

9. The composition of claim 1, wherein the weight ratio of aqueous phase film former to hydrophobically-modified polysaccharide compound is from about 5:1 to about 1:5, preferably about from about 3:1 to about 1:3.

10. The composition of claim 1, wherein the composition is devoid of colorant.

11. A method of making up skin comprising applying the composition of claim 1 to a color cosmetic composition which has been previously applied to skin.

12. A method of making up skin comprising applying the composition of claim 1 to skin, and applying a color cosmetic composition to the composition of claim 1 which has been previously applied to skin.
